(19) United States
(12) Patent Application Publication
(10) Pub. No.: US 2012/0179073 A1
(43) Pub. Date: Jul. 12, 2012

(54) Ischemic Stroke Therapy

(76) Inventor: Henry Nita, Redwood Shores, CA (US)

(21) Appl. No.: 13/424,166

(22) Filed: Mar. 19, 2012

Related U.S. Application Data
(63) Continuation of application No. 11/820,734, filed on Jun. 20, 2007.

(51) Int. Cl. A61N 7/00

(52) U.S. Cl. 601/2

(57) ABSTRACT

A method for delivering ultrasound energy to a patient's intracranial space includes the steps of forming a hole in a patient's skull, locating an ultrasound transmitter near or into the hole, and transmitting ultrasound from the transmitter into the intracranial space, wherein the Mechanical Index of ultrasound energy traveling through cerebral tissue in the intracranial space is less than 1.0, the power intensity delivered to a target tissue in the intracranial space is greater than 50 mW/cm² and less than 200 mW/cm², and the frequency of the transmitted ultrasound is within the range between 500 kHz and 2 MHz. Microbubbles, aspirin, both microbubbles and aspirin, and a mixture of microbubbles and aspirin, can also be delivered into the intracranial space.

### INCORPORATION BY REFERENCE

Applicant expressly incorporates herein by this reference the entire disclosures in pending application Ser. Nos. 11/203,738 filed Aug. 15, 2005, 11/165,872 filed Jun. 24, 2005, 11/274,356 filed Nov. 15, 2005 and 11/490,971 filed Jul. 20, 2006.

### BACKGROUND OF THE INVENTION

1. Field of the Invention

The present invention relates to ischemic stroke therapy, and in particular, to methods for delivering ultrasound energy to a patient's intracranial space.

2. Description of the Prior Art

After the onset of an ischemic stroke, affected blood vessels can leak blood and/or the bloods' constituents into the intra-cerebral space if (i) the occluded vessels are revascularized too late, (ii) the vessels are damaged during the revascularization process, and (iii) the blood vessels are opened too quickly. Bleeding into the intra-cerebral space is a result of a breakdown of the blood brain barrier (BBB) and is also known as a Hemorrhagic Stroke. Such a bleed after the onset of an ischemic stroke can further worsen the patient's clinical sequel and reduce his/her likelihood for recovery. In such a situation, the physician is presented with a conundrum; if the patient is not treated, it is almost guaranteed to result in a permanent deficit for the patient. On the other hand, the treatment options available today are limited to endovascular approaches, which have their own limitations.

Therefore, it is desirable for the physician to have a treatment option that opens the occluded vessels while minimizing the risk for such bleeding or opening up the BBB. The BBB is composed of endothelial cells packed tightly in brain capillaries that more greatly restrict passage of substances from the bloodstream than endothelial cells in capillaries elsewhere in the body.

Processes from astrocytes surround the epithelial cells of the BBB providing biochemical support to the epithelial cells. The BBB is an effective way to protect the brain from common infections. However, during an ischemic stroke, the blood vessels that are affected can become leaky over time or as a result of the treatment protocol.

Endovascular treatment protocols for opening up occluded intracranial blood vessels face access challenges due to the tortuous nature of the intracranial blood vessels. Also, endovascular devices are at risk of causing a vessel perforation during navigation since typical fluoroscopy imaging techniques are inhibited by the occluded vessels not filling during contrast injections. In addition, opening an occlusion using endovascular devices will typically result in instantaneous blood flow to the effected blood vessels. Such a dramatic increase in flow to the effected blood vessels is associated with higher rates of bleeds into the intracerebral space.

### SUMMARY OF THE INVENTION

It is an object of the present invention to provide methods for treating ischemic stroke.

It is another object of the present invention to provide improved methods for delivering ultrasound energy to a patient's intracranial space to treat ischemic stroke.

In order to accomplish the above-described and other objects of the present invention, the present invention provides a method for delivering ultrasound energy to a patient's intracranial space that includes the steps of forming a hole in a patient's skull, providing an access device that enables positioning and locating an ultrasound transmitter near or within the hole, and transmitting ultrasound energy from the transmitter into the intracranial space, wherein the Mechanical Index (MI) of ultrasound energy traveling through cerebral tissue in the intracranial space is less than 1.0, the power intensity delivered to a target tissue in the intracranial space is greater than 50 mW/cm² and less than 200 mW/cm², and the frequency of the transmitted ultrasound is within the range between 500 kHz and 2 MHz.

According to some embodiments of the present invention, the transmitter is advanced into the hole.

According to other embodiments of the present invention, microbubbles, aspirin, both microbubbles and aspirin, and a mixture of microbubbles and aspirin, can be delivered into the intracranial space.

According to other embodiments of the present invention, the transmitter can be manually or automatically maneuvered during the therapeutic delivery of ultrasound energy.

According to yet another embodiment, an acoustically conductive film can be placed between the transmitter and the patient's duramater to provide a sterile barrier and reduce the risk of infection.

### BRIEF DESCRIPTION OF THE DRAWINGS

FIG. 1 is a graphical representation of the relationship between power intensity, mechanical index and frequency for an ultrasound procedure.

FIG. 2 illustrates the sweep angle for an ultrasound probe that is placed above a burr hole in the skull.

FIG. 3 illustrates the sweep angle for an ultrasound probe that is placed through a burr hole in the skull.

FIG. 4a is a cross-sectional view of a human skull and brain showing an access device and an ultrasound device, with the ultrasound device directed to treat one portion of a clotted cerebral artery.

FIG. 4b is a similar view as FIG. 4a showing the ultrasound device redirected to treat a second portion of the clotted cerebral artery.

FIG. 5 is an enlarged view of the access device and ultrasound device of FIGS. 4a and 4b showing an acoustically conductive material located within the burr hole between the ultrasound device and the hole

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

Ultrasound techniques have the advantage of opening up the occluded blood vessels in the brain in a more controlled manner, thereby reducing the risk of hemorrhage stroke when opening up the affected blood vessel. There are three parameters that are important for safe and effective ultrasound treatment of stroke: mechanical index (MI), power intensity, and frequency.

TransCranial Doppler (TCD) technique, which is used routinely for diagnostics, has been shown to safely and effectively lyse clots (Clotbust Trial), but this system is not a commercially viable solution for acute stroke since the technique is limited to treatment of only select intra-cranial vessels due to the dramatic ultrasound attenuation effects of transmitting through the skull. Walnut Corporation used TCD for clinical trials in early 2000 in Germany but instead used lower transmitter frequencies of ˜300 kHz to enable targeting of all intracranial vessels. However, this approach ran into another problem in that the skull thickness variability from patient to patient caused some patients to receive too much energy (if the skull is thinner) in the cerebral space and to produce intracranial bleedings. These bleedings in the Walnut Clinical Trials were associated with ultrasound energy delivered to the intracranial space at the upper threshold of the mechanical index (MI). FDA regulations and guidance for such devices require that the MI≦5 1.9 to prevent bio-effects or damages to the tissue. The mechanical index is an estimate of the maximum amplitude of the pressure pulse in tissue. It gives an indication as to the relative risk of adverse mechanical effects (streaming, cavitations) on the tissue. The FDA regulations allow a mechanical index of up to 1.9 to be used for all applications except ophthalmic, which has a maximum of 0.23.

The present inventors have concluded that since ischemic stroke patients are more susceptible to bleeding from their intracranial blood vessels, due to a breakdown or partial breakdown in the BBB, it is necessary to treat these patients drastically below the allowed MI of 1.9. The present inventors have discovered that the Mechanical Index should be below MI<1, preferably ≦0.08, and most preferably below 0.5. This includes being below these MI numbers for any brain tissue that is exposed to ultrasound energy, including the dura mater, which is a thin tissue layer sandwiched between the surface of the brain cortex and the cranium. Therefore, to treat these patients safely, the brain tissue needs to be treated below the typical safety thresholds while delivering enough energy to lyse the clot. In addition, it is not possible to stay below these suggested MI numbers using TCD due to the variability in the skull thickness and other limitations associated with TCD transmitting through several structures. However, by removing the skull from the ultrasound transmission field, it is possible to safely deliver ultrasound energy to the desired blood vessel and/or affected tissue at a MI that reduces the risk for hemorrhagic stroke while still being effective in aiding in clot lysis.

U.S. Pat. No. 6,716,412 (Unger), U.S. Publication No. U.S. 2005/0124897 (Chopra), and U.S. Pat. No. 7,037,267 (Lipson et al.) disclose using an ultrasound probe through a man-made hole such as a burr hole during a stroke, thereby eliminating the attenuation from the skull. However, there is no description in these references describing the key parameters for safely treating an ischemic brain while effectively lysing the detrimental clot without the skull in place. Also, the FDA regulations and guidance only provide broad limits for the spatial peak time-averaged power intensity (I-SPTA) (720 mW/cm²), and require a MI of less than 1.9. The present inventors have discovered that the only viable recipe for safe and effective treatment is to achieve a power level at the targeted clot site between 20 to 200 mW/cm² while the maximum MI needs to be MI≦1.0, preferably MI≦0.8 and most preferably MI≦0.5. Conventional wisdom might suggest that staying within the FDA regulations and guidelines for power intensity and MI would be adequate to protect stroke patients from adverse effects associated with transmission of ultrasonic energy. However, due to the friable nature of an ischemic brain and the unpredictable nature of the head/skull/brain geometry to transmission of ultrasound energy and the compounding variable of transmitting through multiple tissue types at once, it is necessary to further reduce the maximum power intensity and resultant maximum MI to a lower level that is still effective in clot lyses, and to prevent potentially catastrophic hemorrhages. The parameters proposed above by the inventors for acute stroke therapy are distinctive from the prior art and conventional wisdom, and significantly reduce potential adverse brain tissue bio-effects while enabling effective stroke treatment.

The third critical parameter for safe and effective application of ultrasound stroke therapy through a burr aperture is frequency. William Culp, Jurgen Eggers, George Shaw and others describe that the most effective clot lyses are achieved at lower frequencies than diagnostic ultrasound, preferably between 20 kHz-2 MHz. See: (i) George Shaw, Basic Science of Ultrasound and Clot lysis: Pre-clinical Models. Interventional Stroke Conference, Feb. 2, 2005 New Orleans, (ii) Jurgen Eggers, Guinter Seidel, Bjorn Koch, R. Konig. SonoThrombolysis in acute Ischemic Stroke for patients ineligible for rt-PA. Neurology 2005; 64; 1052-1054, and (iii) William Culp et al. “Intracranial Clot lysis with Intravenous Microbubbles and TCT. Stroke 2004; 35; 2007-2011. Higher frequencies are less efficient in transmitting through tissue due to higher energy attenuation as the ultrasound travels through the respective tissue, resulting in significantly lower energy intensity at the desired locations (which are usually 4-7 cm from the energy source). See (i) Hoogland R. Ultrasound Therapy. Delft, the Netherlands: Enraf-Nonius; 1989, (ii) Low J. Reed A. Electrotherapy Explained, Principals and Practice. Butterworth and Henemann, 3 edition; 2000. (iii) Williams AR. Ultrasound: Biological Effects and Potential Hazards, Ward A.R. Electricity, Fields and waves in Therapy. Marrickville, Australia 1986. Lower frequencies are more effective in lysing clots, such frequencies also travel more efficiently through tissue, thereby increasing the Mechanical Index. Lower frequencies also increase the risk of standing wave phenomena which can result in significantly higher Mechanical Index and unexpected devastating effects in the patents brain tissue. This respective rise in MI is because MI is a standard measure of the acoustic output in an ultrasound system, defined as the peak rarefactional pressure of an ultrasound longitudinal wave propagating in a uniform medium, divided by the square root of the center frequency of the transmitted ultrasound pulse. Therefore, it is necessary to choose a transmission frequency that is effective in lysing the clot without increasing the MI above an unsafe threshold. The present inventors believe that it is advantageous to deliver energy well above the 300 kHz range believed to be optimum for the lysing of clot while safely transmitting through skull and brain tissue.

In order to safely and effectively lyse clots that are approximately 4-7 cm or more from the transducer through a burr hole access or any other aperture in the skull, it is necessary to prescribe an operational algorithm involving the two parameters; power intensity (PI) and frequency (F), whereas the resulting MI for the treatment of acute stroke patients is less than a critical value, preferably M<1. Based on experimental laboratory work, the inventors discovered that the optimal algorithm/scenario to meet such requirements is when:
Power Intensity (PI)at clots: 50 mw/cm²<PI<200 mW/cm²
Frequency (F): 500 kHz<F<2 MHz
The resultant Mechanical Index (MI): 0.2<MI<1.0

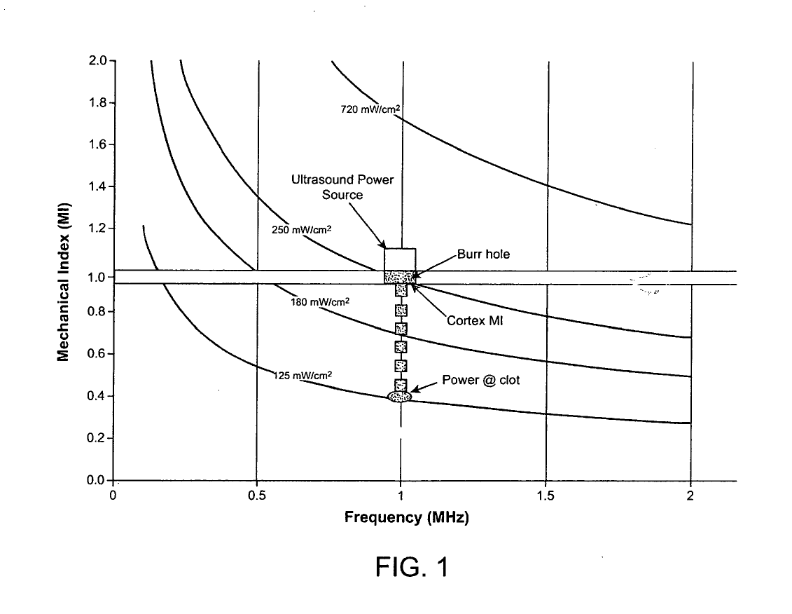

FIG. 1 illustrates for a graphical representation of the relationship between PI, MI and F through a specific example using this algorithm. In this example, it is assumed the ultrasound probe is placed below the top surface of the cranium and within an opening in the skull, while the targeted clot is approximately 5 cm from the probe. This example assumes an attenuation effect of 50% ultrasound energy as a result of transmission through 5 cm of brain tissue with a 1 MHz transmission frequency, assuming ideal coupling between the probe and brain tissue (no or minimal losses). By not transmitting the energy through a variable skull thickness, the attenuation rate is quite predictable at any given frequency through a known brain tissue distance. By starting with a transmission power of approximately 250 mW/cm² the clot will be exposed to a lysing power of approximately 125 mW/cm² at a MI of approximately 0.4. The Power Intensity, power at clots as shown in FIG. 1 can be expanded about this point setting by modulating the transducer power so as to achieve a mechanical index within the brain tissue at range of 0.2<MI<1.0, thereby to achieve a lysing power of 50-200 mW/cm² at different clot locations. This example does not explicitly describe the resulting lysing power at the clot as a result of transmission at frequency other than 1 MHz but within the recommended algorithm of 500 kHz<F<2 MHz.

Another key parameter (in addition to the above three parameters) that is necessary to optimize the procedural efficacy while minimizing trauma to the patient is to minimize the number of required man-made access holes in the skull and/or keep the diameter of the access hole to a minimum while effectively lysing the clot. In addition, since the location of the clot will often only be generally known (i.e. which side of the brain), it is paramount that the approach to the procedure maximizes the sweep angle of the ultrasound probe with respect to the brain tissue (target area), thereby facilitating the most flexibility in finding the location of the clot and/or treating it from a single man-made hole of minimum diameter. For purposes of the present invention, the creation of access holes in the skull, and the delivery of ultrasound energy via the access holes using ultrasound probes as mentioned herein below, can be carried out using any of the techniques and devices disclosed in pending application Ser. Nos. 11/203,738 filed Aug. 15, 2005, 11/165,872 filed Jun. 24, 2005, 11/274,356 filed Nov. 15, 2005 and 11/490,971 filed Jul. 20, 2006.

The inventors have discovered that to maximize the specific sweep angle of the ultrasound probe, it is advantageous to first use a transducer that is slightly smaller than the man-made hole to allow for angulations within the hole, and then place the distal end of the probe below the top surface of the skull. Using within-the-hole angulations technique rather than manipulating the distal end of the probe above the man-made hole significantly reduces the required hole diameter for treating areas over a specific sweep angle. In addition, physically placing the distal end of the ultrasound probe partially within or below the hole allows for more predictable and uniform transmission of power to brain tissue. For example, if the transducer is above the burr hole, extra power is needed to target brain tissue at the edge of the ultrasound beam width due to normal attenuation of a diverging ultrasound beam or attenuation associated with the ultrasound beam clipping the edges of the skull near the man-made hole. Also, extra power is needed because the probe is farther away from the target and the ultrasound energy needs to overcome attenuation losses associated with a longer distance to the target tissue or clot. Therefore, locating the ultrasound probe above the man-made hole, or tightly fitting the transducer within a man-man hole, has several disadvantages over the present approach discovered by the inventors. By locating the transducer below the top surface of the skull and partially within the man-made hole, ultrasound power losses are reduced, resulting in a reduced output power from the transducer and a reduction of brain cortex heating, as well as less potential tissue exposure to ultrasound energy (at higher MI) about the periphery of the ultrasound probe. In addition, the desired power can be safely delivered to the clot site when the probe is located below the top surface of the skull since attenuation through brain tissue is more predictable than the variable attenuation experience by variable bone thickness associated with a probe located above the hole or through the skull.

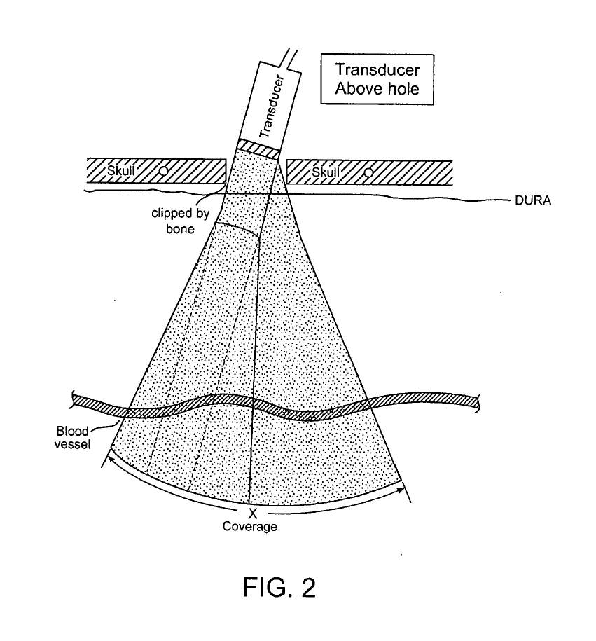

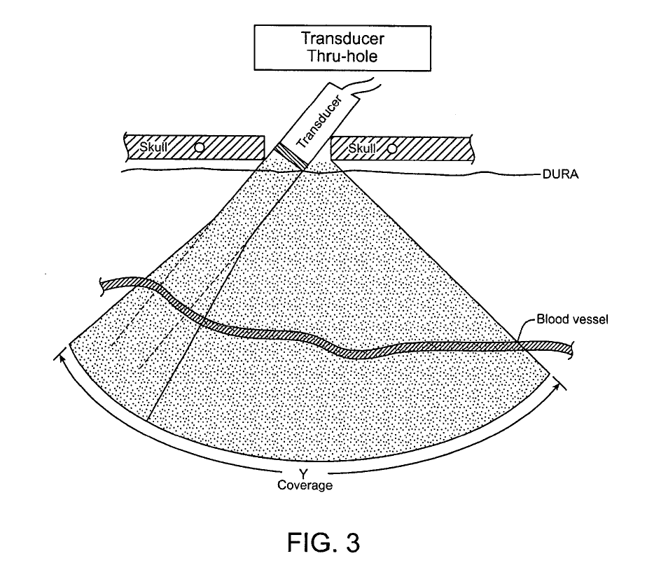

In addition, it would be more advantageous to place at least a portion of the ultrasound probe within or through the aperture and then angulate the distal end of the probe (or a whole probe) at a desirable direction where the clot is located. In this manner, the ultrasound beam coverage area is much larger than if the probe were above the hole for the same hole size. FIGS. 2 and 3 illustrate the sweep angles for ultrasound probe placement through the hole (FIG. 3)—Y-coverage, versus ultrasound probe placement above the hole (FIG. 2)—X-coverage. As can be seen from FIGS. 2 and 3, a greater sweep angle is obtained when the probe is placed within the hole.

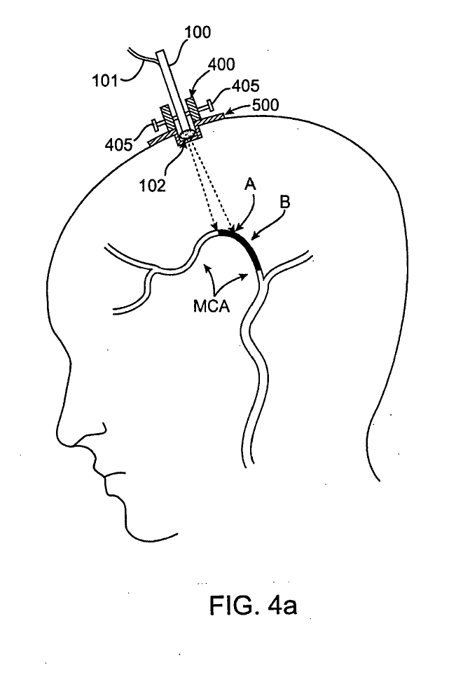

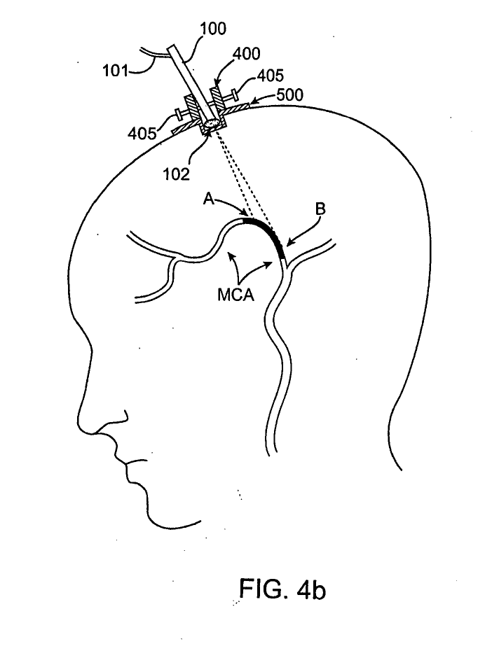

To support the placement of the ultrasound device, an access device 400 may be used as shown in FIG. 4a and FIG. 4b. The access device 400 also has attributes that enable precise positioning and immobilization of the ultrasound device 100 at a specific angle or range of angles with respect to the skull. The access device 400 can be a part of a stereotaxis frame, or it can be frameless and therefore directly secured to the skull. Examples of such frameless devices include the “Navigus System for Frameless Access” and the NAVIGATION™ products made by Image-Guided Neurologics, Inc., located in Melbourne, Fla. Using either a stereotaxis frame or a frameless access device, the ultrasound device 100 may be placed on the scalp surface, on the skull surface, inside the skull, or positioned above the skull. The ultrasound device 100 may also be directed to the treatment area and immobilized at a desired angle, thereby allowing longer therapy time without the risk of disengagement from the treatment target or misdirection by the ultrasound device 100. If the treatment area is of a larger size or length, the access device 400 may allow re-positioning and can be used to immobilize the ultrasound device 100 at various parts of the treatment area. For example, treatment of larger cerebrovascular clots may require that a proximal portion of the clot be targeted and treated first before repositioning the ultrasound device 100 to target and treat a more distal portion of the clot. Alternatively, a large treatment area may be treated by either manually or automatically moving the ultrasound device 100 through a range of angles with respect to the skull or clot. The angles are defined by the pivoting of the ultrasound device 100 about a point on its length with respect to the skull or clot, about any of the three defined orthogonal axes of a rectangular coordinate system. The automated movement range can be restricted by limiting the ultrasound device 100 to a range of angles and then continuously powering the ultrasound device 100 through various angles by a power driven element (such as a motor). For example, in order to treat a cerebral clot which occludes several centimeters of the blood vessel in one or more locations, it may be necessary to have the ultrasound device 100 oscillate over a range of angles to treat the these clots. The angle between the ultrasound device 100 and skull can range from 1 to 179 degrees, and more typically between 45 to 135 degrees. Alternatively, the ultrasound device 100 can be automatically moved without power to the ultrasound device 100 being temporarily turned off. The ultrasound device 100 can be limited to a specific range of angles through (i) a plate (not shown)having a slot placed about the ultrasound device 100, or around the ultrasound device 100, or (ii) other fixtures such as limiting pins (not shown) that could restrict the range of angles. If a plate is used, the plate can be fixed with respect to the base of the access device 400. The orientation and length of the slot would dictate the range of angles the ultrasound device 100 could oscillate through. Alternatively, rather than limiting the angles through a separate device, the drilled hole size would dictate the maximum angles of the transducer. By manually or automatically moving the ultrasound device 100 about a range of angles, it may eliminate the need to precisely identify the location of the clot(s) and the need to specifically target the therapeutic ultrasound to the same location. Instead, it may be possible to lyse a clot located anywhere within that brain hemisphere by simply modulating the angle of the ultrasound device 100 with respect to the skull. No diagnostic imaging would be necessary to first identify the clot location. If the distal end of the ultrasound device 100 is located below the top surface of the skull, then the effective therapeutic angle range is much greater than being above the skull surface. In addition, if the ultrasound device 100 is automatically moved within a range of angles, it may be desirable to control the pattern or path of the ultrasound device 100. Such patterns include linear, circular, random, and any combination thereof. Also, the speed of the ultrasound device 100 being moved could also be controlled, thereby controlling the dose of ultrasound energy to the targeted tissue. In addition, by continuously or intermittently moving the ultrasound device 100 during transmission of the therapeutic ultrasound, it may be possible to temporarily treat tissue with MI values exceeding those disclosed herein without causing an adverse effect.

In one aspect of the present invention, a method for delivering ultrasound energy to a patient's intracranial space involves fixing at least one access device 400 (as shown in FIGS. 4a and 4b) to the patient's skull, advancing at least one ultrasound device 100 at least partway through the access device 400, and transmitting ultrasound energy from the ultrasound device 100 to the patient's intracranial space. The access device 400 may be fixed in place with screws through the scalp and into the skull, or alternatively the scalp may be retracted so that the base of the access device 400 is located directly on the skull.

Another aspect of the present invention includes the provision of a sterile or non-sterile acoustically conductive medium 102 as shown in FIGS. 4a and 4b to facilitate ultrasound energy transmission to the targeted site. The acoustically conductive medium 102 is positioned between the ultrasound device 100 and the patient. The ultrasound device 100 will normally include a transducer (not shown) that emits ultrasound energy. The acoustically conductive medium 102 may include a condense gel, diluted gel, oil, saline or any other semi-solid, fluid or gaseous material that conducts ultrasonic energy. The acoustically conductive medium 102 may also be embodied in the form of a compliant pack which contains any of the above-identified acoustically conductive media inside the pack. In one embodiment, the pack has a thin conductive shell designed to contain the acoustically conductive medium. The compliant pack may be located within the hole in the skull, on the skull surface, on the scalp surface, at the tip of the ultrasound device 100, or inside or under the access device 400. The acoustically conductive medium 102 may be delivered through the transducer or around the transducer, through an additional introducer (not shown) or around the introducer, or through the access device 400, intermittently or continuously during the procedure. Low viscosity fluids may be preferred for this approach and may also assist in cooling of the ultrasound device 100 and/or adjacent tissues (such as the scalp, skull or brain). The acoustically conductive medium 102 may also be located within the hole in the skull, on the skull surface, on the scalp surface, as well as inside the access device 400 and /or inside the introducer.

In another aspect of the present invention, a thin film 500 (or a liner) as shown in FIGS. 4a and 4b can be positioned between the access device 400 and the skull, and/or between the ultrasound device 100 and the skull. The film 500 serves as a sterility barrier between the patient's inner tissue (epidural space) and the access device 400 or the ultrasound device 100. The film 500 can also serve as an acoustically conductive medium to facilitate ultrasound energy transmission, and may aid in the sealing of the burr hole to prevent bleeding of the skull. The film 500 may have thrombogenic properties on its surfaces to enhance thrombosis of the scalp and/or skull bleeding. The film 500 may be attached to the scalp, the skull, the access device 400, the introducer or the ultrasound device 100. The film 500 can be composed of organic or synthetic polymers. The polymer material can be coated or impregnated with oil, gels, saline or other fluids to enhance its acoustically conductive properties. Alternatively, the surfaces of the film 500 can be hydrophilic, thereby attracting fluid and/or ions that would also enhance its conductive properties.

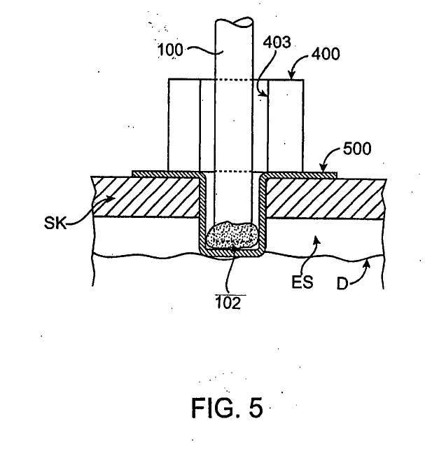

FIG. 4a is a cross-sectional view of a human skull and brain showing the access device 400 and the ultrasound device 100 having electrical cables 101 and targeting one portion of the clotted cerebral artery, treatment area A. Acoustically conductive medium 102 is positioned at the end of the ultrasound device 100 between the ultrasound device 100 and the patient. Stabilizing members 405 surround the ultrasound device 100 to immobilize the ultrasound device 100 within the access device 400 and with respect to the skull and the treatment area A. FIG. 4b shows the access device 400 and the ultrasound device 100 of FIG. 4a being redirected to treat a second portion of the clotted cerebral artery, treatment area B. Stabilizing members 405 are repositioned and immobilize the ultrasound device 100 within the access device 400 with respect to the skull and the treatment area B. FIG. 5 is an enlarged view of FIGS. 4a and 4b, with the access device 400 having an inner channel 403 and being mounted to the skull SK. The ultrasound device 100 is located within the channel 403, and an acoustically conductive material 102 is located within the burr hole between the thin film 500 and the ultrasound device 100. The thin film 500 is located within epidural space ES and sits directly on duramater D. As other alternatives to FIG. 5, an acoustically conductive material 102 can be located inside the burr hole and the ultrasound device 100 within or above the hole, or an acoustically conductive material 102 may be placed within the epidural space ES and directly on duramater D. In addition, a thin film 500 may be located between the patient and an acoustically conductive material 102.

Contrast agents such as microbubbles are often used in conjunction with TCD to help identify intracranial blood vessels or intracranial landmarks. As the ultrasound energy hits the microbubbles, these microbubbles implode, thereby increasing the backscatter from blood to aiding in vessel detection. Also, contrast agents have been used to assist TCD in diagnostics and clot lyses and have been shown to enhance the effect of the ultrasound energy alone. However, when using the microbubbles to assist in therapeutic lyses of clot, it is desirable to reserve or preserve as many microbubbles as possible for the therapeutic step rather than implode all of them during the diagnostic phase. This would require that the diagnostic phase be performed with a minimal amount of microbubbles that are ruptured during this phase by minimizing the amount of power transmitted to the microbubbles below its rupture threshold. Studies have shown that microbubbles can enhance imaging of blood vessels even when the bubbles are not destroyed. However, with standard TCD algorithms, most if not all microbubbles are destroyed during diagnostic imaging because higher energy settings are required to overcome the attenuation associated with transmission through the skull. Therefore, with TCD diagnostics, most microbubbles would be consumed prior to the therapeutic phase. However diagnostics can successfully be performed at lower power levels if bone is removed from the skull (i.e., creating a burr hole), thereby providing an opportunity to preserve the microbubbles for the therapeutic ultrasound phase by not imploding them during the diagnostic phase.

It is important to mention that these contrast agents are used systemically, so they are present in the entire cerebrovascular circulation system. In acute ischemic stroke therapies that use contrast agents (e.g., microbubbles) and ultrasound energy, it is essential to deliver an appropriate amount of ultrasound energy to avoid bio-effects and breakdown in the BBB. In such therapies, it is critical to predictably deliver a required amount of ultrasound energy to clots that is effective in dissolving clots without causing intracranial bleeding, such as through the use of the algorithm set forth above.

Aspirin has also been strongly recommended as a prophylactic approach to avoid acute ischemic stroke and appears to be effective. The inventors have discovered that use of aspirin in combination with other embodiments of the present invention, or in combination with a contrast agent combined with other embodiments of the present invention, may be beneficial in acute ischemic stroke therapies by enhancing the lytic effect of ultrasound on the clot. The aspirin can be taken orally, through intravenous delivery (IV), intra-arterial delivery (IA), as a depository or an alternative delivery technique, either before, during or after the ultrasound treatment.

2b/3a inhibitors have also shown abilities to assist in clot lysis and could be used to help bind microbubbles to the clot. This binding mechanism may assist in the clot lysis process during the delivery of the therapeutic ultrasound. Attaching the microbubbles to the clot will ensure that the energy generated when the microbubble implodes will be imparted on the clot, thereby assisting with breaking up the clot. See Culp C et al. Intracranial Clot Lysis With Intravenous Microbubbles and Transcranial Ultrasound in Swine. 2004—Am Heart Assoc. Stroke. 2004; 35:2407.

The following are a few examples illustrating methods for delivering ultrasound energy to a patient's intracranial space according to the principles of the present invention.

### EXAMPLE 1

At least one hole is formed in the patient's skull, and at least one ultrasound probe is positioned near but preferably at least partially within the hole. Next, microbubbles are delivered through IV or IA delivery. An ultrasound diagnostics procedure is carried out by using low power and sweeping the ultrasound probe (rotating and/or angling and/or moving the probe towards and away from the brain cortex) about or within the hole to locate the clot location in the brain. During this procedure, it is preferable to use ultrasound probe power levels that minimize the number of microbubbles that are imploded to less than 80%. As described above, the diagnostic algorithm comprises Power Intensity (PI) at clots: 50 mw/cm²<PI<200 mW/cm², and Frequency (F): 500 kHz<F<2 MHz with the resultant Mechanical Index (MI): 0.2<MI<1.0. Also, the power level used for the diagnostic treatment will either be equivalent to or lower than the value used during the therapeutic phase. Once the clot is located, the probe guide system (as described in pending applications Ser. No. 11/203,738 and Ser. No. 11/274,356) is fixed to that specific angle or angle range. Next, a therapeutic ultrasound procedure is performed where the remaining intact microbubbles are available to assist in lysing the clot aging using an algorithm comprises Power Intensity (PI) at clots: 50 mw/cm²<PI<200 mW/cm², Mechanical Frequency (F): 500 kHz<F<2 MHz with a resultant Index (MI): 0.2<MI<1.0.

### EXAMPLE 2

At least one hole is formed in the patient's skull, and at least one ultrasound probe is positioned near but preferably at least partially within hole. Next, microbubbles and aspirin are delivered sequentially, through any known delivery method, including orally, or through IV or IA. Next, an ultrasound diagnostics procedure is performed in the manner described above for Example 1 to locate a clot, and then therapeutic ultrasound is delivered to the clot in the manner described above for Example 1.

### EXAMPLE 3

At least one hole is formed in the patient's skull, and at least one ultrasound probe is positioned near but preferably at least partially within hole. Next, a mixture of microbubbles and aspirin is delivered, through any known delivery method, including orally, or through IV or IA. Next, an ultrasound diagnostics procedure is performed in the manner described above for Example 1 to locate a clot, and then therapeutic ultrasound is delivered to the clot in the manner described above for Example 1.

### EXAMPLE 4

At least one hole is formed in the patient's skull, and at least one ultrasound probe is positioned near but preferably at least partially within hole. The transmitter is moved within the hole to select a transmission angle, and then ultrasound is transmitted from the transmitter into the intracranial space along the transmission angle towards a clot. The transmitter can then be moved within the hole to select a second transmission angle, and ultrasound is then transmitted from the transmitter into the intracranial space along the second transmission angle towards another clot. Optionally, the agents and respective algorithms described in Examples 1 through 3 could also be employed in combination with this specific example.

### EXAMPLE 5

At least one hole is formed in the patient's skull, and at least one ultrasound probe is positioned near but preferably at least partially within hole. The transmitter is moved within the hole to select a transmission angle, and then ultrasound is transmitted from the transmitter into the intracranial space along the transmission angle towards a clot. The moving and transmitting steps are then repeated to transmit ultrasound substantially throughout the patient's intracranial space without removing the transmitter from the hole. Optionally, the agents and respective algorithms described in Examples 1 through 3 could also be employed in combination with this specific example.

### EXAMPLE 6

At least one hole is formed in the patient's skull. A support and access device 400 (as shown in FIGS. 4a and 4b and described in detail in pending application Ser. No. 11/274,356) is attached to the patient's skull adjacent the hole. At least one ultrasound transmitter/probe is advanced through and into the hole, and then moved within the hole to select a transmission angle. Ultrasound is transmitted from the transmitter into the intracranial space along the transmission angle towards a clot, with the transmitter being supported by the support and access device during the advancing, moving and transmitting steps. Optionally, the agents and respective algorithms described in Examples 1 through 3 could also be employed in combination with this specific example.

### EXAMPLE 7

At least one hole is formed in the patient's skull. A support and access device 400 as shown in FIGS. 4a and 4b is attached to the patient's skull adjacent the hole. At least one ultrasound transmitter/probe is advanced through and into the hole, and automatically moved within the hole at selected path. Ultrasound is transmitted from the transmitter into the intracranial space along the transmission path towards a clot, with the transmitter being angulated within the support and access device 400 during the procedure. Additional steps, such as advancing and moving the transmitter either automatically or manually, may be implemented as well. Optionally, the agents and respective algorithms described in Examples 1 through 3 could also be employed in combination with this specific example.

While the description above refers to particular embodiments of the present invention, it will be understood that many modifications may be made without departing from the spirit thereof. The accompanying claims are intended to cover such modifications as would fall within the true scope and spirit of the present invention.

1-8. (canceled)

9. A method for delivering ultrasound energy to a patient's occluded intracranial blood vessel comprising:
forming a hole in a patient's skull;
placing acoustically conductive medium on the epidural brain tissue inside the hole
advancing an ultrasound transmitter into the hole;
transmitting ultrasound energy within frequency range 500 KHz-2 MHz from the transmitter to treat to the occluded intracranial blood vessel, and:
wherein the Mechanical Index of uRrasound energy delivered through intracranial tissue in the intracranial space is less than 1.0 and power intensity delivered to the occluded vessel in the intracranial space is greater than 50 mW/cm²,

10-12. (canceled)

13. The method of claim 9 wherein conductive medium is selected from the group consisting of a condense gel, diluted gel, saline and a material that conducts ultrasonic energy.

14. The method of claim 9, further including:
angling the transmitter within the hole to select a transmission angle; and
transmitting ultrasound energy from the transmitter to the occluded intracranial blood vessel along the transmission angle.

15. The method of claim 14, further including performing the angling and transmitting steps repeatedly to transmit ultrasound energy throughout the patient's intracranial space without removing the transmitter from the hole.

16. The method of claim 14, further including transmitting ultrasound energy from the transmitter to the occluded intracranial blood vessel along additional transmission angles.

17-18. (canceled)

19. The method of claim 16, wherein movement of the transmitter within the hole to select a transmission angle is along a defined path.

20. The method of claim 19, wherein the path is circular, linear or a combination thereof.

21. The method of claim 9, further including delivering thrombolytic agents to the patient.

22-23. (canceled)

24. A method for delivering ultrasound energy to a patient's occluded intracranial blood vessel, comprising:
forming a hole in a patient's skull;
placing acoustically conductive medium on the epidural brain tissue inside the hole
locating an ultrasound transmitter near the hole;
transmitting ultrasound energy from the transmitter to the occluded intracranial blood vessel, and;
wherein ultrasound energy frequency is in range 500 KHz-2 MHz and power intensity delivered to the occluded vessel in the intracranial space is less than 200 mW/cm².

25. The method of claim 24, wherein the locating step includes the step of advancing the transmitter into the hole.
